(19) United States
(12) Patent Application Publication
(10) Pub. No.: US 2004/0018556 A1
(43) Pub. Date: Jan. 29, 2004

(54) Reagent and method for determination of a substance using an immunoaggregator

(76) Inventor: Thomas L. Cantor, El Cajon, CA (US)

Correspondence Address: Peng Chen, Morrison & Foerster LLP, Suite 500, 3811 Valley Centre Drive, San Diego, CA, 92130-2332, US

(21) Appl. No.: 10/209,162

(22) Filed: Jul. 29, 2002

(51) Int. Cl. G01N 33/53; G01N 33/574

(52) U.S. Cl. 435/7.1

(57) ABSTRACT

The present disclosure relates to reagents and methods useful for analyzing for the presence or amount of a particular analyte in a sample. Such reagents and methods are particularly useful in that false positive and false negative results are suppressed.

### TECHNICAL FIELD

The present disclosure relates to reagents and methods useful for analyzing for the presence or amount of a particular analyte in a sample. Such reagents and methods are particularly useful in that false positive and false negative results are suppressed.

### BACKGROUND

The sensitive determination of components, capable of being bound immunologically, such as polyvalent antigens (peptides, proteins, polysaccharides, viruses, bacteria, specific cells) using two or optionally more antibodies, which are directed against spatially different antigen determinants, is known as an immunoradiometric or immunoenzymometric sandwich assay (two-site immunoassay). The most common method for carrying out this determination is where the target antigen (or polyvalent antigen) is incubated with a first antibody, which either may be bound in solid phase to a suitable carrier material such as sepharose, agarose, plastic tubes, etc., or present homogeneously, for example, biotinylated, in solution; and the addition of a predetermined quantity of a second or more labeled antibodies in liquid phase. The second antibody and optionally additional antibodies preferably is of a specificity for a different binding site on the target antigen than the first antibody in order to exclude competition between the antibodies for the same binding sites or binding sites with spatial proximity to each other on the target antigen. Such steric hindrance competition would interfere with the functionality and sensitivity of the test. The first labeled antibody, which may be bound to the solid phase or, for example, biotinylated, as well as the second or additional labeled antibody present in solution are generally added in excess. The target antigen may then be determined based on the label activity fixed to the first antibody, or which remains in solution and is not bound immunologically. If a heterophilic antibody were to bind to both of these assay antibodies there could be an interference with the test caused by crossing bridging the labeled antibody to the unlabelled antibody. This cross bridging of labeled antibody to unlabelled antibody would give the appearance of higher than actual levels of endogenous antigen. This would constitute a false positive result.

Monovalent analytes, e.g., steroids, ligands, drugs, prions, can be assayed immunologically by a competitive immunoassay. The most common example of a competitive immunoassay is where the target antigen is incubated with a primary antibody, which either may be bound in solid phase to a suitable carrier material such as sepharose, agarose, plastic tubes, etc., or present homogeneously, but, which may be separated from the solution at the completion of the immunoassay reaction (for example, by an immunological precipitation reaction with centrifugation and decantation). In addition, there is added to the reaction solution a labeled analyte, e.g., by radioactivity or an agent capable of producing a chemiluminescent signal, etc. As the primary antibody is present in a limited quantity with a limited number of binding sites, there is a competition between the added labeled antigen and the unknown quantity of endogenous antigen. At the end of the immunoassay reaction the primary antibody is separated from the solution which may be accomplished by removal of the solid phase to which it is bound from the solution or the addition of an agent which renders the primary antibody to come out of solution (for example, by addition of a second precipitating antibody that is directed against the primary antibody which can be followed by centrifugation and decantation). If a heterophilic antibody were to bind to the primary antibody there could be an interference with the test which might interfere with the binding of the primary antibody to the labeled antigen. This decreased binding of labeled antigen to the primary antibody would give the appearance of higher than actual levels of endogenous antigen. This would constitute a false positive.

Complete monoclonal IgGs, especially those derived from monoclonal antibodies, or their immunologically reactive fragments (Fab or F(ab′)₂) are generally used for the immunological sandwich assays due to their particular specificities. However, polyclonal antibodies may also be used. Generally, polyclonal antibodies are used for competitive immunoassays, but monoclonal antibodies have also been used.

Although specific antibodies may be used in these two-site immunoassays and competitive immunoassays, human serum samples frequently contain substances which lead to nonspecific reactions. These nonspecific reactions frequently occur. These types of reactions lead to wrong test results with correspondingly serious consequences for therapeutic measures. The occurrence of nonspecific reactions can be attributed to substances (interferents including, e.g., heterophilic antibodies) present in a test sample, which, like the target analytes, will become bound to the test reagents. Generally, for sandwich immunoassays, these interfering factors bind to the immunoreactant at a different site than the analyte being detected, but still lead to the formation of complexes even in the absence of analyte. Generally, for competitive immunoassays, these interfering substances prevent the binding of the labeled antigen with the primary antibody leading to a false positive result.

Heterophilic antibodies in a patient's serum can lead to serious consequences. For example, heterophilic antibodies may interact with the antibody used in the assay. This interaction would typically lead to assay interferences which may produce false positive or false negative results which may then lead to an incorrect diagnosis. As provided by the College of American Pathologists, “Endogenous human heterophilic antibodies which have the ability to bind to immunoglobulins of other species are present in the serum or plasma of more than 10% of patients.”

Heterophilic antibodies may develop resulting from different exposures, such as rheumatoid arthritis, vaccinations, influenza, animal contact (pets), allergies, special diets, (e.g., cheese,), blood transfusions, alternate animal, contact therapy, (e.g., thymic cells, sheep cells, embryonic cells), autoimmune diseases, dialysis, patent medicines, (OKT3), maternal transfer, cardiac myopathy and G.I. disease (E. coli).

In sandwich assays, heterophilic antibodies may cause false positive results by cross bridging a given capture and label assay antibodies, thus making it falsely appear that the analyte being tested for is present in a given sample.

In competitive immunoassays the reduction of labeled antigen bound to the primary antibody is interpreted as a larger amount of endogenous unknown antigen. Therefore, if a heterophilic antibody binds to the primary antibody and interferes with the binding of the primary antibody to the labeled antigen this would cause false positive results.

To avoid these interfering reactions, nonspecific non-aggregated immunoglobulins or immunoglobulin fragments (generally IgG) or heat or chemically aggregated immunoglobulins or immunoglobulin fragments (generally IgG) of the same animal species, from which the specific assay antibodies originate, are frequently added in excess to such immunoassays. See Addison, G. M., Radioimmunoassay and Related Procedures in Medicine, 1:131-147 (1974). The use of specific non-aggregated polyclonal antibodies, requires large amounts of nonspecific mouse IgG in the form of mouse serum, mouse ascites or isolated mouse immunoglobulin to achieve interference-suppression (approximately 300-500 μg/mL; Boscato LM, et al., Clin. Chem., 32:1491-1495 (1986)). However, the preparation of mouse IgG on the required kilogram scale is difficult with the presently available methods under economically interesting conditions and may be considered critical from ethical points of view.

Other related efforts have been directed at the patient where heterophilic antibodies are targeted prior to undertaking a given assay. Such methods have involved immunosuppressant therapy (using, e.g., Cyclosporine A), antibody fragment administration, humanized and chimeric antibody administration (using mouse CDR and human framework), and pegylation of the heterophilic antibodies. See U.S. Pat. No. 5,614,367; Boscato et al., Clin Chem., 43:27-33 (1988); and Kahn et al., J Clin. Endocrin. Metabolism., 66:526-33 (1988). These methods are, however, invasive and risk stimulating the patient's immune system which might lead to auto immune disease. In addition, immunosuppressive therapy sets the patient at risk of infection and liver and kidney toxicities.

Another attempt to eliminate the problems associated with interfering factors has involved the humanization of antibodies. In this approach genetic engineering techniques have been used to combine mouse complementary determining regions with human framework and constant regions or human constant regions and mouse framework. However, the resulting IgG molecule is still potentially antigenic and an immune response will produce human anti-human antibodies. See U.S. Pat. No. 5,614,367; and Kricka, Clin. Chem., 45(7):942-956 (1999).

A still further method of dealing with the problems encountered by interfering factors is provided in U.S. Pat. No. 4,914,040, which describes the use of an aggregate of antibody to compensate for interfering factors. The aggregate is purportedly formed by cross-linking nonspecific IgGs with one another or with antibody fragments by the action of heat or chemicals.

Another method designed to avoid interference in immunoassays of this type is the use of Fab or F(ab′)₂ fragments for at least one of the specific antibodies used in the immunoassay. This approach blocks the Fe portions of the IgG (rheumatoid factors, anti-Fc immunoglobulins such as IgM) and inhibits the interfering factors from binding the immunoreactants and, thereby, creating false positive or false negative results.

However, interferences from these factors continue to plague immunoassays for a variety of samples despite the use of Fc-free specific antibody reagents. These continued interferences may be attributed to substances in a particular sample, which are directed to Fab or F(ab′)₂. These can be removed in appropriate immunoassays by the addition of native or aggregated Fab or F(ab′)₂ fragments, which are not specific for antigen that is to be determined (See European Patent No. 0,083,869). According to EP 0,083,869, completely nonspecific IgGs, in native as well as in aggregated form, bring about no interference-suppressing effect in the immunoassays reviewed. The method of blocking using non specific IgGs, however, purportedly has the significant disadvantage that, considerable amounts of high-grade and nonspecific immunoglobulin reagents are required. See U.S. Pat. No. 4,914,040. According to this method, for example, at least 100 μg/mL of aggregated, nonspecific Fc-free immunoglobulin fragments, which have proven to be effective, may be used to suppress interferences.

The object of the invention is therefore to suppress or eliminate interferences in an assay, e.g., the interfering effects of heterophilic antibodies, and, thus, to allow for a lower cost and more accurate analysis of analytes.

### SUMMARY OF THE INVENTION

The present disclosure relates to reagents and methods useful for analyzing an analyte, particularly in a sample containing interfering factors. The reagents and methods, as further described below, generally involve the steps wherein an immunoreactant is provided that specifically binds to an analyte in a sample; and an aggregate is provided which suppresses a false positive or a false negative signal caused by an interferent (if present) in the sample. The aggregate is generally made up of a plurality of protein components, which are derived from an organism. Although these protein components bind to interfering factors, they do not specifically bind to the analyte. Further, the protein components are aggregated together by an immuno-aggregator that specifically binds to the protein components, thus the contemplated aggregates are formed without chemical crosslinking or heat treatment.

In a particular aspect of the present disclosure, the immunoreactant, the protein component or the immuno-aggregator may be a polyclonal antibody, a monoclonal antibody, or a fragment thereof. Frequently, antibody fragments may be Fab or F(ab′)₂ fragments. In addition, antibodies may be selected from a specific type of immunoglobulin, for example, representative antibodies may be selected from the group consisting of IgA, IgD, IgE, IgG and IgM. When the antibody is an IgG, such antibody may be selected from the group consisting of IgG₁, IgG₂, IgG₃, and IgG₄.

The plurality of protein components may frequently be made up of whole IgGs or IgG and Fab or F(ab′)₂ fragments. Moreover, frequently the plurality of protein components may be protein components from the same or different species. In addition, the protein components and the immunoreactant may be derived from the same or different species. The protein components, when they are derived from different species, may be derived from different vertebrate species. Frequently the protein components of the present disclosure may be derived from a mammal. Representative mammals of the present disclosure, frequently may be selected from the group consisting of bovine, goat, sheep, equine, rabbit, guinea pig, murine, human, feline, porcine, monkey and dog. Also, frequently the protein components may be derived from the same types/subtypes or different types/subtypes of immunoglobulins.

Also, frequently the analyte and the immunoreactant may be derived from the same or different species. For example, the analyte may be derived from a human sample, the protein components may be murine proteins, e.g., mouse proteins, and the immuno-aggregator may be a goat anti-murine protein antibody, e.g., a goat anti-mouse protein antibody.

In one aspect, aggregates of the present disclosure may have a concentration ranging from about 0.1 μg/ml to about 5,000 μg/ml of the immunoassay reaction solution. These aggregates may also be measured by molecular weight, and frequently such aggregates have a molecular weight above about 320,000 daltons. For example, such aggregates may have a molecular weight ranging from about 320,000 daltons to about 100 million daltons.

Frequently, in reagents and methods of the present disclosure, the molar ratio between protein components in the aggregate and the immuno-aggregator in the aggregate is more than 1, e.g., 2, 3, 4, 5, 6, 7, 8, 9 or 10.

Aggregates of the present disclosure may comprise intact IgGs, or intact IgGs and Fab or F(ab′)₂ fragments. Frequently, these aggregates may further comprise a water soluble macromolecule. In one aspect, water soluble macromolecules of the present disclosure may be selected from the group consisting of a water soluble protein, a water soluble polysaccharide and a water soluble polymer.

In another aspect of the present disclosure the immunoreactant may carry a label. Such immunoreactants may also be attached to a surface suitable for conducting an immunoassay. Further, reagents of the present disclosure may be comprised of one or more immunoreactants, e.g., two different immunoreactants, which can sandwich an analyte. In this circumstance, occasionally both of the two different immunoreactants may be intact IgGs. Relatedly, on occasion, one of the two different immunoreactants may be an intact IgG and the other immunoreactant may be a Fab or F(ab′)₂ fragment.

In one aspect, when the two different immunoreactants are murine, e.g., mouse, anti-human analyte antibodies or fragments thereof, the protein components in the aggregate may be non-specific murine, e.g., mouse, antibodies or fragments thereof, and the immuno-aggregator in the aggregate may be a goat anti-murine, e.g., goat anti-mouse, antibody or fragments thereof.

Reagents of the present disclosure may also optionally contain a reaction accelerator, a detergent, or a stabilizer, or a combination thereof.

In one embodiment of the present disclosure, the presently described reagents may further comprise an analyte bound to the immunoreactant in the reagent.

In another embodiment, the present disclosure provides kits for analyzing an analyte, which kits may comprise at least the reagents described hereinabove, which kit may be provided in a suitable container. Occasionally, in kits of the present disclosure the immunoreactant and the aggregate are contained in the same or different containers. In yet another embodiment, kits of the present disclosure may also comprise a buffer and/or an instruction for using the reagent to analyze an analyte. The contemplated kits include those kits with a regulatory approval for its indicated use, e.g., clinical diagnosis.

The present disclosure further relates to methods for analyzing an analyte wherein such methods may comprise the contact of an analyte in a sample with a reagent of the type described above, under suitable conditions to allow binding between the analyte, if present, in the sample, and the immunoreactant in the reagent. This binding may occur while false positive or false negative signals, caused by an interferent (if present) in the sample, are suppressed. This suppression may occur via an interaction between the interferent and the aggregate in the reagent, or via an interaction between the aggregate and a non-specific binding site for the interferent. In addition, in accordance with these methods, binding between said analyte and said immunoreactant may be assessed to analyze the presence or amount of the analyte in the sample.

In reagents and methods of the present disclosure, the immunoreactant and the protein components in the aggregate may occasionally be derived from the same species.

The present methods are useful for analyzing a variety of analytes, for example analytes from the group consisting of a cell, a cellular organelle, a virus, a molecule and an aggregate or complex thereof. Representative cells may be, e.g., animal cells, plant cells, fungus cells, bacterium cells, recombinant cells or cultured cells. Representative cellular organelles may be, e.g., nuclei, mitochondrion, chloroplasts, ribosomes, ERs, Golgi apparatus, lysosomes, proteasomes, secretory vesicles, vacuoles or microsomes. Representative molecules may be, e.g., inorganic molecules, organic molecules or complexes thereof. Representative organic molecules may be, e.g., amino acids, peptides, proteins, nucleosides, nucleotides, oligonucleotides, nucleic acids, vitamins, monosaccharides, oligosaccharides, carbohydrates, lipids or complexes thereof. Frequently, analytes may be selected from the group consisting of a hormone, a cancer marker, a steroid, a sterol, a pharmaceutical compound, a metabolite of a pharmaceutical compound and a complex thereof.

A variety of types of samples may be analyzed for a particular analyte or combination thereof. For example, frequently the sample may be a mammalian sample. When the sample is derived from a mammal, such mammal may be, e.g., a bovine, goat, sheep, equine, rabbit, guinea pig, murine, human, feline, monkey, dog or porcine sample. Occasionally, samples of the present disclosure may be clinical samples, or human clinical samples in particular. Clinical samples may be body fluid samples or other non-fluid samples, such as, serum, plasma, whole blood, sputum, cerebral spinal fluid, amniotic fluid, urine, gastrointestinal contents, hair, saliva, sweat, gum scrapings or tissue from biopsies.

Reagents and methods of the present disclosure generally inhibit interferents from interfering with analysis for a particular analyte. Generally, the aggregate in the reagent substantially suppresses a false positive or a false negative signal caused by an interferent, if present, in a sample. In one aspect, such interferents may be, e.g., a heterophilic antibody, a rheumatoid factor, a lipoprotein, a fibrin, a clotting factor, an IgE, a human antibody to allergens, a human anti-mouse immunoglobulin, a human anti-goat immunoglobulin, a human anti-bovine immunoglobulin, a human anti-dog immunoglobulin and a human anti-rabbit immunoglobulin, etc.

Reagents and methods of the present disclosure can be used to suppress false positive and false negative results in any suitable immunoassay formats. In one aspect, reagents and methods of the present disclosure are used to suppress false positive and false negative results in sandwich or competitive assay formats.

For example, in sandwich assays, heterophilic antibodies may cause false positive results by cross bridging a given capture reagent and label assay antibodies, thus making it appear that the analyte being tested for is present, or present in a greater amount, in a given sample. The false positive result caused by the heterophilic antibodies can be suppressed by the reagents of the present disclosure, which interact with the heterophilic antibodies and suppress cross bridging activities of the heterophilic antibodies.

In another example, in competition assays, heterophilic antibodies may cause false positive results by binding to the assay antibody and blocking the binding between the assay antibody and the labeled analyte, thus making it appear that the endogenous analyte being tested for is present, or present in a greater amount, in a given sample. The false positive result caused by the heterophilic antibodies can be suppressed by the reagents of the present disclosure, which interact with the heterophilic antibodies and suppress the binding between the heterophilic antibodies and the assay antibody.

Frequently, binding between the analyte and the immunoreactant is assessed by a format selected from the group consisting of, e.g., an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, chemiluminescence assay, lateral flow immunoassay, immuno radio metric assay (IRMA), μ-capture assay, linear flow membrane chromatography, inhibition assay, energy transfer assay, avidity assay, turbidometric immunoassay and time resolved amplified cryptate emission (TRACE) assay. Reagents and methods of the present disclosure can also be used to suppress false positive and false negative results in these immunoassay formats.

The present disclosure also provides a method of forming an aggregate suitable for assaying an analyte, which method comprises: a) providing a plurality of protein components, wherein said protein components do not specifically bind to an analyte to be analyzed; and b) aggregating, without chemical crosslinking or heat treatment, said protein components with an immuno-aggregator that specifically binds to said protein components under suitable conditions into an aggregate of a defined size suitable for assaying said analyte.

### BRIEF DESCRIPTION OF THE DRAWINGS

FIG. 1 illustrates an example wherein a heterophilic antibody causes a false positive in a sandwich assay and blocking with immunoaggregated IgG.

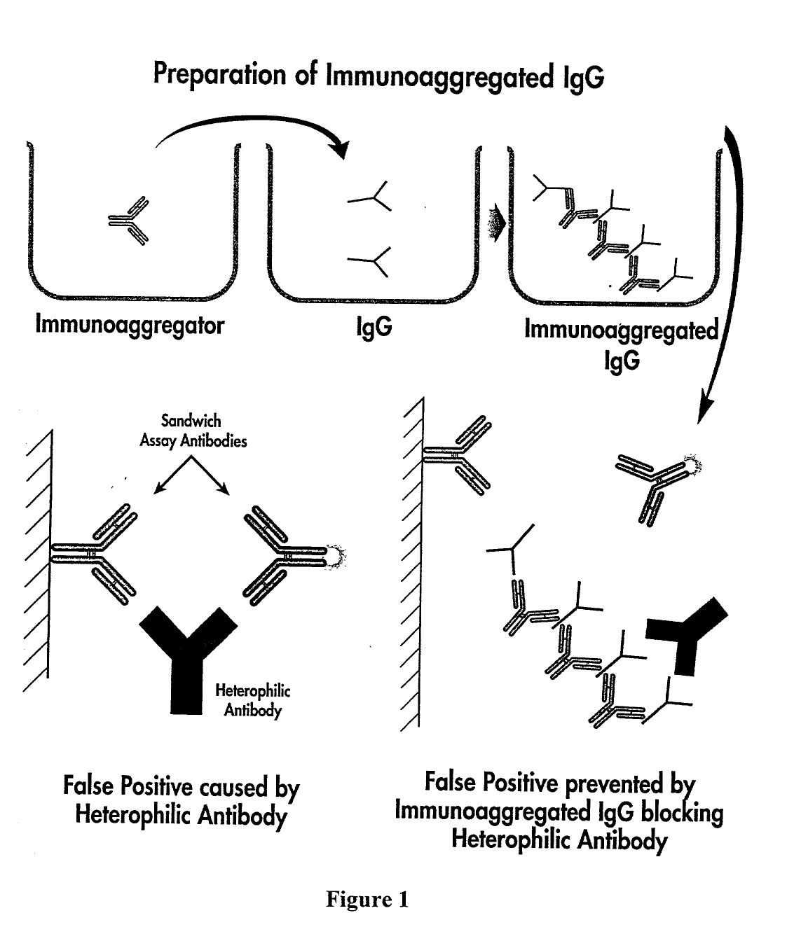

FIG. 2 illustrates an example wherein a heterophilic antibody causes a false negative in a sandwich assay and blocking with immunoaggregated IgG.

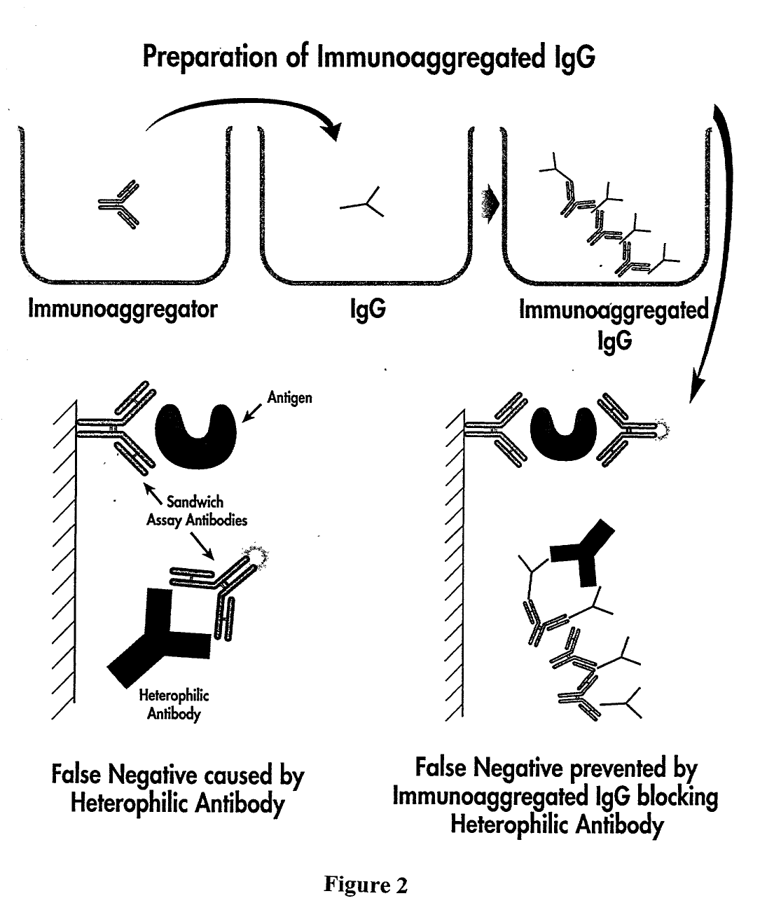

FIG. 3 illustrates an example wherein a heterophilic antibody causes a false positive in a competitive assay and blocking with immunoaggregated IgG.

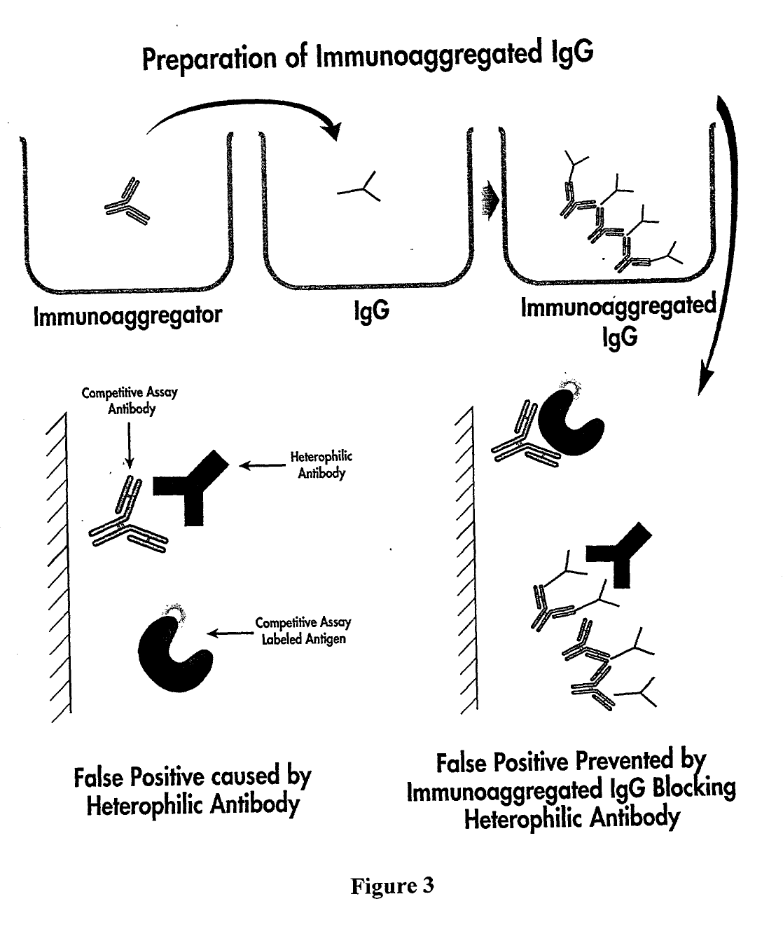

FIG. 4 illustrates an example wherein non-specific binding causes a false positive in a sandwich assay and blocking with immunoaggregated albumin.

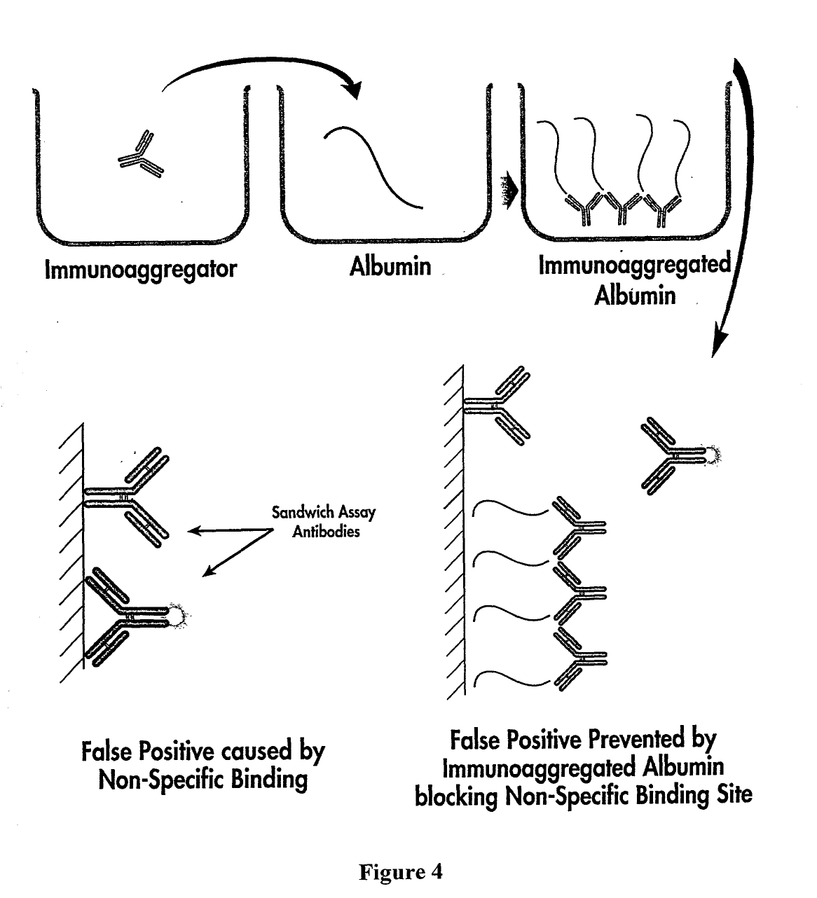

FIG. 5 illustrates an example wherein non-specific binding causes a false negative in a competitive assay and blocking with immunoaggregated albumin.

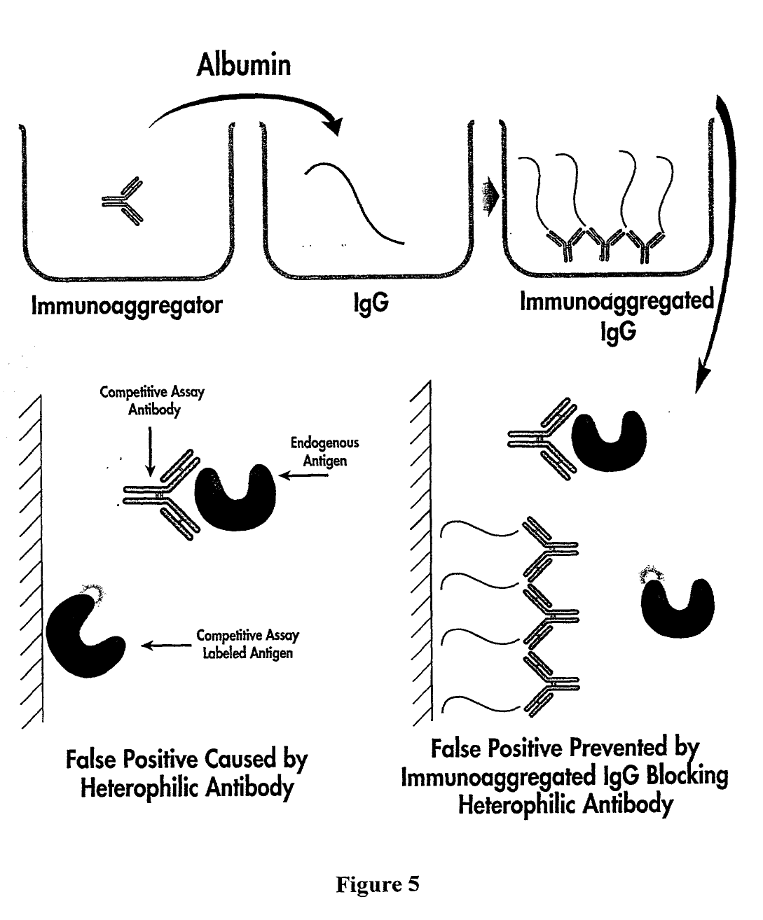

FIG. 6 illustrates an example wherein heterophilic antibody causes a false negative in an antibody detection assay and blocking with immunoaggregated IgG.

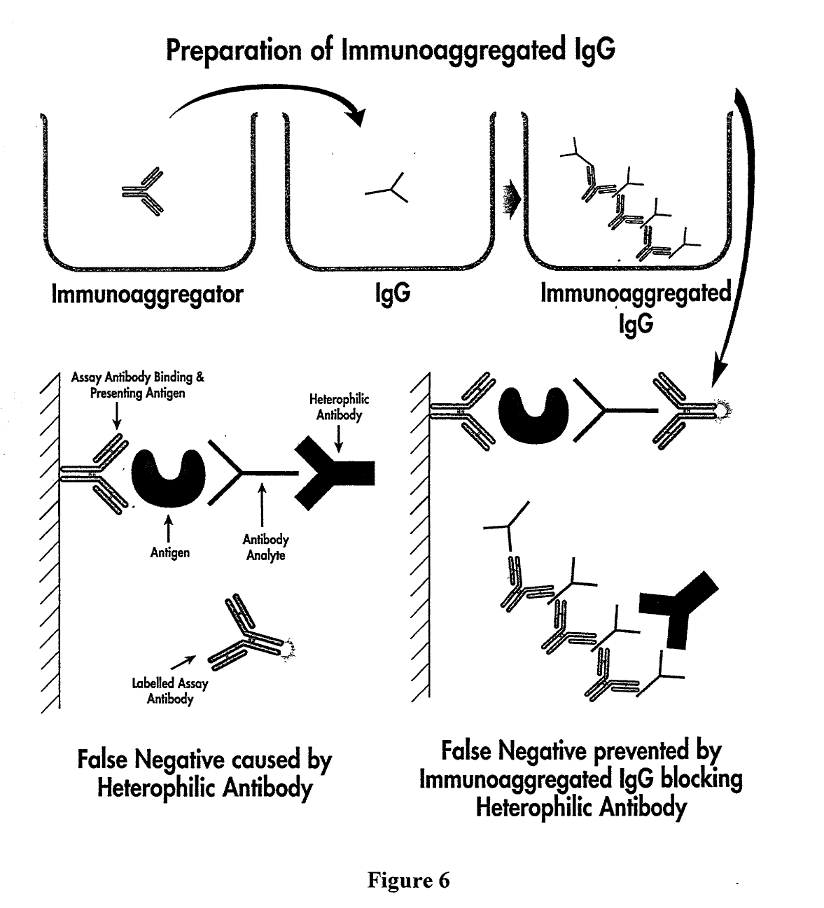

FIG. 7 illustrates an example wherein heterophilic antibody causes a false positive in an antibody detection assay and blocking with immunoaggregated IgG.

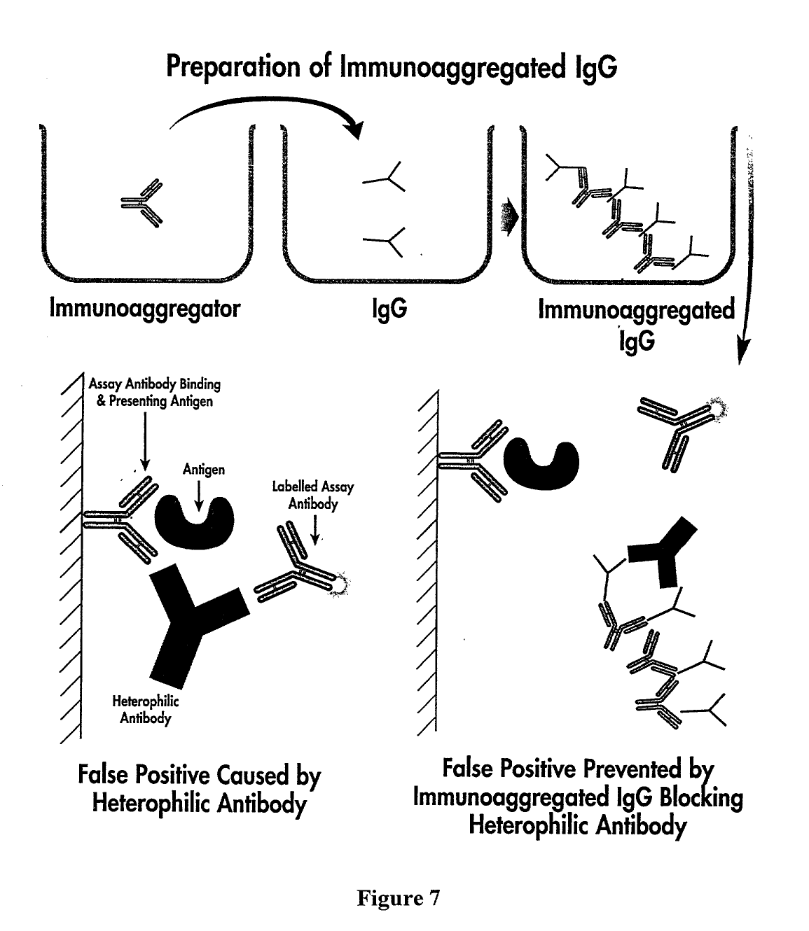

### DETAILED DESCRIPTION OF THE INVENTION

The present disclosure relates to reagents and methods useful for analyzing an analyte, particularly in a sample containing interfering factors. The reagents and methods, as further described below, generally involve the steps wherein an immunoreactant is provided that specifically binds to an analyte in a sample; and an aggregate is provided which suppresses a false positive or a false negative signal caused by an interferent (if present) in the sample. The aggregate is generally made up of a plurality of protein components, which are derived from an organism. This organism may be the same or different than that from which the sample, immunoreactant and immuno-aggregator are obtained from. Although these protein components may bind to interfering factors or to places where an interfering factor may bind, they do not specifically bind to the analyte. Further, the protein components are aggregated together by an immuno-aggregator that specifically binds to the protein components, thus the contemplated aggregates are formed without chemical crosslinking or heat treatment.

A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in patents, applications, published applications and other publications that are herein incorporated by reference, the definition set forth in this section prevails over the definition that is incorporated herein by reference.

As used herein, “a” or “an” means “at least one” or “one or more.”

As used herein, “immunoreactant” generally refers to an immunoglobulin or fragments or derivatives thereof. Such immunoreactants are preferably specific for, or may specifically bind to, a particular analyte.

As used herein, an “immunoglobulin” refers to any protein with immunoglobulin-like domains, i.e., a complex of two heavy chains and two light chains. Antibody is a type of an immunoglobulin. However, an immunoglobulin as used herein, refers to the action of a binding protein and can be a non-antibody molecule such as MHC molecules and some cell adhesion molecules and cytokines receptors.

As used herein, “antibody” refers to specific types of immunoglobulin, i.e., IgA, IgD, IgE, IgG, e.g., IgG₁, IgG₂, IgG₃, and IgG₄, and IgM. An antibody can exist in any suitable form and also encompass any suitable fragments or derivatives. Exemplary antibodies include a polyclonal antibody, a monoclonal antibody, a Fab fragment, a Fab′ fragment, a F(ab′)₂ fragment, a Fv fragment, a diabody, a single-chain antibody and a multi-specific antibody formed from antibody fragments.

As used herein, “a diabody” refers to a double chain antibody formed by association of two single chain antibodies, each single chain antibody comprising a heavy chain variable domain, a linker and a light chain variable domain. In such diabodies, the heavy chain of one single-chain antibody binds to the light chain of the other and vice versa, thus forming two identical antigen binding sites (see Hollinger et al., Proc. Natl. Acad. Sci. USA, 90:6444-6448 (1993); Carter & Merchan, Curr. Op. Biotech., 8:449-454 (1997); and U.S. Pat. No. 6,420,113).

As used herein, “immuno-aggregator” refers to an immunoglobulin, or a fragment or derivative thereof, that specifically binds to the protein components to form the aggregate without chemical crosslinking or heat treatment. Preferably, an immuno-aggregator is a polyclonal antibody, a monoclonal antibody or fragments thereof.

As used herein, “a plurality of protein components” refer to a plurality of proteins that are aggregated together by an immuno-aggregator to form an aggregate without chemical crosslinking or heat treatment. The protein components can be multiple units or molecules of the same proteins. Alternatively, the protein components can be single or multiple units or molecules of different proteins. The protein components can be immunoglobulins, e.g., antibodies, or fragments or derivatives thereof. Alternatively, the protein components can be non-immunoglobulin proteins, e.g., BSA, casein, ovalbumin, lactose, etc. In one aspect, the protein components may be derived from the same types/subtypes or different types/subtypes of immunoglobulins. These protein components may be protein components from the same or different species. Such protein components, when they are derived from different species, may be derived from different vertebrate or mammal species.

As used herein, “aggregate” refers to a plurality of protein components that are aggregated together by an immuno-aggregator to form an aggregate without chemical crosslinking or heat treatment. Such an aggregate can be used to suppress false positive or false negative signals in an assay caused by an interferent. The aggregate can be “an aggregate of defined size,” or aggregates with different sizes.

As used herein, “an aggregate of a defined size suitable for assaying said analyte,” means that the largest difference of molecular weight or diameter among the aggregates is less than 50% of the average or median molecular weight or diameter of the aggregates. Preferably, the largest difference of molecular weight or diameter among the aggregates of “an aggregate of a defined size” is less than 40%, 30%, 20%, 10%, 5%, 4%, 3%, 2%, or 1% of the average or median molecular weight or diameter of the aggregates. Also preferably, the aggregates of “an aggregate of a defined size” have the same molecular weight or diameter.

As used herein, the term “suppress” is meant to refer to varying degrees of removal or reduction of false positive or false negative signals caused by an interferent. For example “complete” suppression is meant to refer to about 100% removal of such signals or lack of signals arising from either false positive or false negative interferences. In addition, substantial suppression means about at least 70%, preferably means at least 80%, more preferably at least 90%, and most preferably at least 95% above removal of false positive or false negative signals caused by an interferent.

As used herein the term “false positive” is meant to refer to an analytical result indicating that a certain analyte is present, when such analyte is not present, or that a certain analyte is present in an amount greater than its actual amount.

As used herein the term “false negative” is meant to refer to an analytical result indicating that a certain analyte is not present, when such analyte is present, or that a certain analyte is present in an amount less than its actual amount.

As used herein the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein the term “heterophilic antibody” is meant to refer to one example of potential interferents in an immunoassay and, as such, to one type of interferon that said invention is able to remove. Generally, these antibodies are circulating antibodies which cross-react with proteins of another species. Further, heterophilic antibodies producing false positive or false negative results may exist in sufficient quantities or concentrations to interfere with the results of a given assay due to interaction with proteins, antibodies, and/or antibody fragments used in these assays.

As used herein the term “assessing” is intended to include quantitative and qualitative determination of an analyte present in the sample, and also of obtaining an index, ratio, percentage, visual or other value indicative of the level of the analyte in the sample. Assessment may be direct or indirect and the chemical species actually detected need not of course be the analyte itself but may for example be a derivative thereof or some further substance.

B. Reagents

Reagents of the present disclosure may generally include the following components, an immunoreactant that specifically binds to an analyte in a sample; and an aggregate which suppresses a false positive or a false negative signal caused by an interferent if present in the sample, the aggregate comprising a plurality of protein components, derived from an organism, wherein the protein components do not specifically bind to the analyte and the protein components are aggregated together by an immuno-aggregator that specifically binds to the protein components, and wherein the aggregate is formed without chemical crosslinking or heat treatment. These reagents are useful for the methods of the invention described below.

Further, in one aspect, the present invention contemplates the reagent described above wherein the immunoreactant is bound to an analyte in the sample.

1. Immunoreactants

The immunoreactants of the present disclosure may be a polyclonal antibody, a monoclonal antibody or a fragment thereof. Further, such immunoreactants may be immunoglobulins, immunoglobulin fragments, and/or Fab or F(ab′)₂ fragments. In one aspect, when the immunoreactant is a polyclonal or monoclonal antibody it may be selected from IgA, IgD, IgE, IgG and IgM. Frequently, the polyclonal or monoclonal antibody immunoreactant is intact IgG. However, in a related aspect, when the immunoreactant is IgG, it may be selected from IgG₁, IgG₂, IgG₃, and IgG₄.

In one aspect of the present invention, one or more immunoreactants are provided which are capable of sandwiching an analyte. For example, the one or more immunoreactants may be useful in a two-site immunoassay where one of the immunoreactants immobilizes the analyte of interest and another immunoreactant binds to the analyte to create a sandwich. Frequently the second immunoreactant may carry a label, as described below. In a related aspect, when two immunoreactants are used, on occasion they may be intact IgGs. Further, one immunoreactant may be intact IgG and the other immunoreactant may be a Fab or F(ab′)₂ fragment. In a particular embodiment the two different immunoreactants may be murine anti-human analyte antibodies or fragments thereof, the protein components in the aggregate may be non-specific murine antibodies or fragments thereof, and the immuno-aggregator in the aggregate may be a goat anti-murine antibody or fragments thereof.

Frequently the immunoreactants are derived from a species other than that from which the sample to be analyzed and the analyte originates. Generally, the immunoreactants are derived from mammal species, such mammal species may be, for example, bovine, goat, sheep, equine, rabbit, guinea pig, murine, human, feline, porcine, monkey or dog. The immunoreactants can also be derived from non-mammalian vertebrate species, e.g., chicken antibodies or chicken IgY antibodies.

The immunoreactant is useful for analyzing a particular analyte in an immunoassay. Frequently, a particular immunoreactant may be attached, adsorbed or otherwise immobilized to a surface suitable for conducting an immunoassay (described below). In a particular related embodiment the immunoreactant may carry a label. Exemplary labeling moieties include chemical, enzymatic, radioactive, fluorescent, fluorescence-quenching, luminescent and fluorescence resonance energy transfer (FRET) labels.

2. Immunoaggregates

Aggregates of the present disclosure are comprised of a plurality of protein components. Aggregates of IgGs which are not specific for the analyte are preferred. Especially preferred is a protein aggregate comprising nonspecific IgG, or fragments thereof and a further protein component. Nonspecific IgGs are preferred which originate from the same animal species as at least one of the specific immunoreactants. Suitable protein components are, e.g., IgGs, and Fab or F(ab′)₂ fragments.

Frequently aggregates are used comprising an IgG molecule and a Fab or F(ab′)₂ fragment, the IgGs as well as the Fab or F(ab′)₂ fragments may originate from the same species of animal as one of the specific immunoreactants.

Further, representative immunoaggregates of the present disclosure include water soluble macromolecules. Examples of water soluble macromolecules include, e.g., a water soluble protein, a water soluble polysaccharide or a water soluble polymer. See U.S. Pat. No. 4,914,040.

The concentration of aggregate required for successful blocking/suppression of interference may vary. However, concentrations of aggregates generally ranging from about 0.1 to about 5000 μg/mL of immunoassay reaction mixture are sufficient, depending on the individual sample for analysis. However concentrations of from about 5 to about 3000 μg/mL and frequently concentrations ranging from about 5 to about 25 μg/mL are used.

In accordance with a particular aspect of the present disclosure, IgG aggregates or IgG/Fab or IgG/F(ab′)₂ aggregates with molecular weights of at least about 320,000 daltons, are used to compensate for interferents in subjects and subject samples. Moreover, the aggregates may contain an IgG of the species from which at least one of the immunoreactants originates, and further, may frequently belong to the same subclass as at least one of the immunoreactants. In one aspect, Preferably, aggregates are used with molecular weights of from about 320,000 to about 100 million daltons. In a further aspect, two-site immunoassays are provided with one or more immunoreactants which are specific for a particular analyte, to which nonspecific IgG aggregates or IgG/Fab or IgG/F(ab′)₂ aggregates with molecular weights greater than 320,000 daltons are added (to compensate for interferents), the IgGs and Fab or F(ab′)₂ fragments contained in the nonspecific aggregates being monoclonal, polyclonal and from the same or different species and same or different types/subclass as the immunoreactants.

Immunoaggregates of the present disclosure may be prepared by conventional methods, for example, by immunoprecipitation or immunoaggregation. Although further and more particularly illustrated in the Example section, a representative immunoaggregate may be formed by contacting a predetermined amount of immuno-aggregator to a solution containing a particular or plurality of protein components. Generally the immuno-aggregator is chosen which will specifically bind to the protein components to form a complex or aggregate. In a particularly preferred aspect, the molar ratio between the protein components and the immuno-aggregator in the aggregate forming solution is more than one. Further, in the aggregate formed by the above process preferably the ratio between the protein components and immuno-aggregator is more than one. Frequently, the molar ratio between the protein components and the immuno-aggregator may be about 2, about 3, about 4, about 5, about 6, about 7, about 8, about 9, about 10, about 11, about 12, about 13, about 14, about 15 or about greater than 15 depending on the binding characteristics and affinity of the immuno-aggregator against the protein components. Thus, the molar ratio of protein components to immuno-aggregator may vary over a particular range. Such a range is useful to ensure that there are no available unoccupied binding sites on the immuno-aggregator after aggregate formation. This molar ratio will also depend on the affinity and binding capacity of the particular immuno-aggregator used.

In one aspect of the present disclosure, the immunoaggregates should not bind with the analyte or immunoreactant, or interfere with the binding between the immunoreactant and the analyte, if present, in a particular sample. These immunoaggregates, however, specifically suppress false positive or false signals which may otherwise be caused by an interferent, if present, in a particular sample. Such suppression may be complete or substantial. As provided above, complete suppression means that about 100% of the false positive or false negative results are suppressed. Substantial suppression means that about at least 70%, preferably means at least 80%, more preferably at least 90%, and most preferably at least 95% or above removal of false positive or false negative signals caused by an interferent.

The false positive or false negative signal may result due to interaction between an interferent and an immunoreactant. In circumstances when a false positive signal is caused, the interferent interacts with the immunoreactant in a way which results in an indication that a particular analyte is present, when, in fact, it may not be present or not present to the extent indicated by the sum of the true positive signal and false positive signal. If the concentration of a particular analyte is sought out, false positive-type interference may produce a signal indicating a high concentration of the particular analyte, when, in fact, the analyte may be present in very small concentrations or not at all. Conversely, a false negative interference may result via an interaction between an interferent and an immunoreactant which results in an indication that a particular analyte is not present, when, in fact, it may be present or a false negative interference may be a signal that does not correspond to the quantity of the analyte present to the extent indicated by the sum of the true positive signal and false negative interference. Similar converse results may occur when the concentration of a particular analyte is sought out as a result of the presence of interferents.

3. Protein Components

The plurality of protein components comprises at least a portion of the immunoaggregates of the present disclosure. These protein components may be a polyclonal antibody, a monoclonal antibody or a fragment thereof. Further, these components may be immunoglobulins, immunoglobulin fragments, and/or Fab or F(ab′)₂ fragments. In one aspect, when the protein components are polyclonal or monoclonal antibodies they may be selected from IgA, IgD, IgE, IgG and IgM. Frequently, the polyclonal or monoclonal antibody protein components are IgG. However, in a related aspect, when the protein components are IgG, they may be selected from IgG₁, IgG₂, IgG₃, and IgG₄.

Frequently these components are derived from a species other than that from which the sample to be analyzed originates. In addition, the protein components and the immuno-aggregator may originate from the same or different species as the immunoreactants. Further, one protein component may originate from the same or different species or be comprised of the same or different immunoglobulins as another protein component in a plurality of protein components. When the protein components comprise different immunoglobulins, they may be made up of different types or subtypes of immunoglobulins or immunoglobulins from different species. When the protein components are derived from different species, they are generally selected from different vertebrate and/or mammal species. Generally, when the protein components are derived from mammal species, such mammal species may be, for example, bovine, goat, sheep, equine, rabbit, guinea pig, murine, human, feline, porcine, monkey or dog protein components. The protein components may also be derived from non-mammalian species such as chicken or even other lower organisms.

4. Immuno-Aggregators

The immuno-aggregator may be a polyclonal antibody, a monoclonal antibody or a fragment thereof. Further, the immuno-aggregator may be an immunoglobulin, immunoglobulin fragment, and/or Fab or F(ab′)₂ fragment. In one aspect, when the immuno-aggregator is a polyclonal or monoclonal antibody it may be selected from IgA, IgD, IgE, IgG and IgM. Frequently, the polyclonal or monoclonal antibody protein components are IgG. However, in a related aspect, when the immuno-aggregator is IgG, it may be selected from IgG₁, IgG₂, IgG₃, and IgG₄.

Frequently the immuno-aggregator is derived from a species other than that from which the sample to be analyzed originates. In addition, the immuno-aggregator is frequently derived from the same species as the immunoreactant. However, the immuno-aggregator may also be derived from a different species than the immunoreactant.

The immuno-aggregator is generally derived from a mammalian species. Generally, when the immuno-aggregator is derived from mammal species, such mammal species may be, for example, bovine, goat, sheep, equine, rabbit, guinea pig, murine, human, feline, porcine, monkey or dog. The immuno-aggregator may also be derived from non-mammalian species such as chicken.

The immuno-aggregator, which specifically binds to the protein components, may preferably originate from a species different than the protein components. For example, the protein components may be murine proteins and the immuno-aggregator may be a goat anti-murine protein antibody.

The immuno-aggregator may be used in a non purified form, such as antisera or in a purified form such as purified by various means including, but not limited to, salt precipitation, alcohol precipitation as in Cohn fractionation, ion exchange, hydrophobic interaction chromatography, affinity purification, molecular weight gel filtration, dialysis or ultrafiltration, sub micron membrane filtration, etc.

5. Assorted Reagents and Diagnostic Means

Aside from the protein components, immunoreactants and immuno-aggregator, the reagents and diagnostic means may contain suitable buffer systems and other optional auxiliary substances such as reaction accelerators, detergents or stabilizers. As suitable buffer systems, 20 to 60 mM of phosphate buffer (pH 7.0) or a 50 mM HEPES/100 mM NaCl buffer system (pH 7.4) may, for example, be used. Representative reaction accelerators such as dextran sulfate, polyethylene glycol, or other reaction accelerators known in the art (with a molecular weight of 6,000 to 40,000) are contemplated. Suitable detergents may include Triton X 100, Tween 20 or pluronic F 68, and other detergents known in the art. Further, suitable stabilizers may include phenol, oxypyrion chloracetamide, methiolate, and other stabilizers known in the art.

The diagnostic means of the invention, when in the form of a solution that optionally is buffered to the desired pH, preferably contains all reagents required for the test. Optionally, stability of the reagents may be increased if the reagents are divided into two or more solutions, which solutions are mixed at the time of analysis. In this connection, it is immaterial whether the aggregates are added separately in a suitable buffer system and/or with the one or more immunoreactants.

The reagents and diagnostic means may be present in the form of a solution or of a dry chemical reagent absorbed onto a surface suitable for conducting an immuno assay.

To produce the diagnostic means in the form of a test strip, an absorptive carrier, preferably filter paper, cellulose or a nonwoven plastic material which are normally used to produce test strips, is impregnated with solutions of the required reagents, in the presence of volatile solvents, such as water, methanol, ethanol or acetone. This process may be accomplished in one more steps.

In another aspect, an open film can be used to produce the diagnostic means in the form of a test strip. Aside from the film-forming agents and pigments, this open film may contain immunoreactants, aggregates, a suitable buffer system and other additives normally used for diagnostic means.

Test papers and test films of the present invention may be used as such or glued in a known manner to support films or preferably sealed between plastic materials and fine-mesh networks, as described in U.S. Pat. No. 3,802,842 and brought into contact with a sample to be investigated (for example, blood, plasma, serum, etc.).

C. Methods

The present disclosure further provides methods for analyzing an analyte, which methods are useful in conjunction with the reagents described above. An illustrative example of the present methods include contacting an analyte in a sample with a reagent described above under suitable conditions to allow binding between the analyte, if present in said sample, and the immunoreactant in the reagent, while suppressing a false positive or a false negative signal caused by an interferent, if present in the sample, via an interaction between the interferent and the aggregate in the reagent, or via an interaction between the aggregate and a non-specific binding site for the interferent; and assessing binding between the analyte and the immunoreactant(s) to analyze the presence or amount of the analyte in the sample.

The analyte can be contacted with the immunoreactant and the aggregate in the reagent simultaneously or sequentially.

In one example, the interferent is a non-analyte moiety that can, nevertheless, bridge between two or more immunoreactants to generate a false positive signal. In this case, the aggregate suppresses the false positive signal by interacting with the interferent, thus suppressing the interaction between the interferent and the various immunoreactants.

In another example, the interferent is a surface that can bind with the immunoreactant(s) to generate a false positive signal. In this case, the aggregate suppresses the false positive signal by interacting with the surface, thus suppressing the interaction between the surface and the immunoreactant(s).

In still another example, the interferent is a non-analyte moiety that can, nevertheless, bind to the immunoreactant to generate a false positive result by preventing a labeled antigen or a labeled antigen analog from binding to the immunoreactant. In this case, the aggregate suppresses the false positive signal by interacting with the interferent, thus suppressing the interaction between the interferent and the immunoreactant.

In yet another example, the interferent is a surface that can bind with the labeled antigen to generate a false negative signal. In this case, the aggregate suppresses the false negative signal by interacting with the surface, thus suppressing the interaction between the surface and the labeled antigen.

In yet another example, the interferent is a non-analyte moiety that can, nevertheless bind with a human antibody, wherein the human antibody is the analyte to be detected in the assay. This binding to the human antibody is in such a manner as to interfere with the binding of a labeled immunoreactant. Such an interference results in a false negative response. In this case, the aggregate suppresses the false negative signal by interacting with the interferent thus suppressing the interaction between the interferent and the labeled immunoreactant.

In yet another example, the interferent is a non-analyte moiety that can, nevertheless bind with an antigen that is a component of the assay reagents. This binding to the assay antigen may be in a cross bridging manner, wherein, an additional binding might occur to the labeled immunoreactant. Such a cross bridging in an assay designed to detect human antibodies would constitute a false positive. In this case, the aggregate suppresses the false positive signal by interacting with the interferent thus preventing the cross bridging.

1. Analytes

A variety of analytes are contemplated in the present disclosure, for example, the analyte may be selected from a cell, a cellular organelle, a virus, a molecule or an aggregate or complex thereof. Generally, such analytes may be categorized under the following criteria, for example, a hormone, a cancer marker, a steroid, a sterol, a pharmaceutical compound, a metabolite of a pharmaceutical compound or a complex thereof. When the analytes are cells, the cell may be, e.g., animal cells, plant cells, fungus cells, bacterium cells, recombinant cells or cultured cells. When the analyte is a cellular organelle, such cellular organelle may be, e.g., a nuclei, a mitochondrion, a chloroplast, a ribosome, an ER, a Golgi apparatus, a lysosome, a proteasome, a secretory vesicle, a vacuole or a microsome. When the analyte is a molecule, such molecule may be, e.g., an inorganic molecule, an organic molecule or a complex thereof. Then the molecule is an organic molecule, such molecule may be, e.g., an amino acid, a peptide, a protein, a nucleoside, a nucleotide, an oligonucleotide, a nucleic acid, a vitamin, a monosaccharide, an oligosaccharide, a carbohydrate, a lipid or a complex thereof.

2. Assays

As provided hereinabove, a variety of immunoassays are contemplated for use in the presently described methods. Generally, however, the object of any given assay is to analyze the binding between an analyte, if present in a sample, and one or more immunoreactants. This analysis may be in sandwich assay or competitive assay format or antibody detection assay format. Representative assays may include, for example, an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, chemiluminescence assay, lateral flow immunoassay, μ-capture assay, inhibition assay, energy transfer assay, avidity assay, turbidometric immunoassay and time resolved amplified cryptate emission (TRACE) assay.

Further, the present invention is suitable for determining all antigens with at least one antigenic determinant. Nonlimiting examples of one-sight and two-site assays where heterophilic antibodies may interfere and potentially produce a false positive result may be directed to analyzing the following analytes: AFP (Bussar-Maatz R, et al., Urologe. A., 32:177-82 (1993)), beta-human choriogonadotropin (β-hCG), cancer antigen 125 (CA 125) (Turpeinen et al., Clin. Chem., 41:1667-9 (1995); Turpeinen et al., Clin. Chem., 36:1333-8 (1990); Boerman et al., Clin. Chem., 36:888-91 (1990); and Reinsberg et al., Clin. Chem., 36:164-7 (1990)), CA 19-9, carcinoembryonic antigen (CEA) (Morton et al., Arch. Surg., 31:1242-6 (1988); Kuroki et al., J. Immunol. Methods, 180:81-91 (1995); and Kricka et al., Clin. Chem., 36:892-4 (1990)), creatine kinase-MB, cortisol, total creatine kinase (CK), erythropoietin, estradiol, free thyroxine, follicle-stimulating hormone (FSH), hepatitis B surface antigen, human choriogonadotropin (hCG) (Cole, Gyneco. Onco., 71:325-9 (1998); Cole, Clin. Chem., 45:313-4 (1999); and Vladuti et al., JAMA, 248:2489-90 (1982)), lueteinizing hormone, myoglobin, osteocalcin, parathyroid hormone (PTH), progesterone, prolactin (Dericks-Tan et al., Klin. Wochenschr., 62:265-73 (1984); and Hellthalar et al., Geburtsh. Frauenheilkd., 55:M55-6 (1995)), prostate specific antigen (PSA) (Stowell et al., Forensic Sci. Int., 50:125-38 (1991)), rebella-specific IgM, thyroxine, thyroglobuline, triiodothyronine, troponin I, and thyroid stimulating hormone (TSH).

In another example, the present invention is suitable for determining a cancer marker (See Table 1 below).

TABLE 1
Consequences of reporting false positives in cancer

| Percentage of Cancers from | Test(s) Used for Cancer Detection Which |
|---|---|
| 100% of all Cancers | Could Yield False Positives |
| Lung 27% | ACTH, PTH, SCC, NSE, CEA, CYFRA, |
| | Prolactin, Renin |
| Liver 2% | Somatomedin-C |
| | AFP, CA19-9, CEA |
| Pancreas 4% | CA19-9, Gastrin |
| Colorectal 12% | CA19-9, Gastrin, CEA |
| Multiple Myeloma 1% | β2-microglobulin |
| Prostate 3% | PSA, PAP |
| Testicle 2% | SCC, AFP, hCG |
| Melanoma <1% | 5-5-Cysteinyl dopa |
| ENT 2% | SCC, CEA |
| Thyroid 1% | hCT, TG, TPA, CEA, NSE |
| Breast 18% | CA15-3, CEA |
| Stomach 12% | CA72-4, CA19-9, CEA, Gastrin |
| Ovarian 7% | CA125, CA19-9, CA72-4 |
| Trophoblast <1% | hCG |
| Corpus Uteria 4% | SCC, CEA, CA125 |
| Cervix Uteria 4% | SCC, CEA, CA125 |

The present invention is also suitable for determining all antigens using an antibody detection assay. For the present reagents and methods can be used in an antibody detection assay for an HIV immunoglobulin antibody using a second antibody, e.g., a second IgG or IgM.

D. Interferents

The presently described reagents and methods are useful for inhibiting an interferent from interacting and binding with immunoreactants utilized in numerous assays. Such inhibition will improve the accuracy of the results of a variety of assays (see “Assays” section above) by substantially suppressing or eliminating false positive or false negative results cause by interferents, if present, in a sample. Interferents contemplated in the present disclosure extend beyond mere heterophilic antibodies and also may include, e.g., a rheumatoid factor, a lipoprotein, a fibrin, a clotting factor, an IgE, a human antibody to allergens, a human anti-mouse immunoglobulin, a human anti-goat immunoglobulin, a human anti-bovine immunoglobulin, a human anti-dog immunoglobulin and a human anti-rabbit immunoglobulin.

Generally, interfering factors (interferents) such as heterophilic antibodies can arise from iatrogenic and noniatrogenic causes. The former may result from the normal response of the human immune system to an administered “foreign” protein antigen. The use of diagnostic or pharmaceutical reagents may lead to the introduction of such proteins and subsequent generation of such antibodies. For example, mouse monoclonal antibodies are foreign proteins in humans and in vivo they may trigger an immune response to produce human anti-mouse antibodies. In many circumstances where mouse monoclonal antibodies have been administered to subjects, those subjects have developed human anti-mouse antibody response. See e.g., Table 2 (Kricka, Clin. Chem., 45(7):942-956, 944).

TABLE 2
Anti-animal Response to Monoclonal Antibodies

| | | | No. Patients | |
|---|---|---|---|---|
| | | | Developing | |
| | | | Antibodies | |
| Monoclonal | Specificity | Condition | (dose) | |
| Mouse | | | | |
| B-E8 | Interleukin-6 | Metastatic renal | 9 | of 112 |
| | | carcinoma | | |
| OKT3 | CD3 | Organ transplant | 695 | of 12,133 |
| | | Cardiac allograft | | |
| | | Cardiac transplant | | |
| B4 | CD19 | B-Cell malignancy | 8 | of 55 |
| BW 4 | Platelet | Thrombosis | 0 | of 4 |
| BW 250/183 | Granulocyte | inflammation | 1 | of 20 |
| BW 431/26 | CEA | colorectal carcinoma | 29% | of 141 |
| BW 494 | Pancreatic | ▪ Pancreatic ductal | 150 | of 150 |
| | carcinoma- | carcinoma | | |
| | associated | ▪ Pancreatic | 8 | of 8 |
| | glycoprotein | carcinoma | | |
| CCR 086 | Mucin | colorectal carcinoma | 4 | of 5 (20 mg), |
| | | | 0 | of 5 (5 mg) |
| CD21 and | CD 21, CD | epstein-barr | 0 | of 1 |
| | | virus-induced | | |
| CD24 | 24 | lymphoproliferative | | |
| | | syndrome | | |
| EMD 55,900 | Epidermal | malignant gliomas | 1 | of 16 |
| | Growth | | | |
| | Factor | | | |
| | receptor | | | |
| IMMU-4 | CEA | colon and rectal | 2 | of 210 |
| | | carcinomas | | |
| LL2 | B cells | Noh-hodgkin | 3 | of 8 |
| | | lymphoma | | |
| Lym-1 | B cells | B-cell malignancy | 2 | of 10 |
| MN-14 | CEA | CEA-producing | 9 | of 18 |
| | | tumors | | |
| NP-4 | CEA | small volume | 5 | of 6 |
| | | tumors | | |
| NR-M1-05 | Melanoma | malignant | 69% | of 20 |
| | Antigen | melanoma | | |
| OKB7 | B cells | Non-hodgkin | 5 | of 18 |
| | | lymphoma | | |
| XMMEN- | Bacterial | bateremia | 3 | of 9 |
| OE5 | endotoxin | | | |
| | lipid A | | | |
| 13G2a | GD-2 | neuroectodermal | 16 | of 18 |
| | | tumors | | |
| 30.6 | Anti-colon | colorectal carcinoma | 10 | of 10 |
| | cancer | | | |
| 96.5 | p97 and 48.7 | melanoma | 4 | of 5 |
| | proteoglycan | | | |
| | melanoma | | | |
| | antigen | | | |
| Rat | | | | |
| YTH 24.5 | CD 45 | Renal | 2 | of 40 |
| 33B3.1 | CD 25 | Bone marrow | 0 | of 15 |

E. Samples

Any suitable samples can be analyzed using the present method. Preferably, a biosample is analyzed using the present method. More preferably the test sample is a clinical sample, more preferably a human clinical sample. Test samples can include a biosample of plant, animal, human, fungal, bacterial and viral origin. If a sample of a mammalian or human origin is analyzed, the sample can be derived from a particular tissue or organ. Exemplary tissues include connective, epithelium, muscle or nerve tissue. Exemplary organs include eye, annulospiral organ, auditory organ, Chievitz organ, circumventricular organ, Corti organ, critical organ, enamel organ, end organ, external female genital organ, external male genital organ, floating organ, flower-spray organ of Ruffini, genital organ, Golgi tendon organ, gustatory organ, organ of hearing, internal female genital organ, internal male genital organ, intromittent organ, Jacobson organ, neurohemal organ, neurotendinous organ, olfactory organ, otolithic organ, ptotic organ, organ of Rosenmüller, sense organ, organ of smell, spiral organ, subcommissural organ, subfornical organ, supernumerary organ, tactile organ, target organ, organ of taste, organ of touch, urinary organ, vascular organ of lamina terminalis, vestibular organ, vestibulocochlear organ, vestigial organ, organ of vision, visual organ, vomeronasal organ, wandering organ, Weber organ and organ of Zuckerkandl. Preferably, samples derived from an internal mammalian organ such as brain, lung, liver, spleen, bone marrow, thymus, heart, lymph, blood, bone, cartilage, pancreas, kidney, gall bladder, stomach, intestine, testis, ovary, uterus, rectum, nervous system, gland, internal blood vessels, etc, are analyzed.

Mammalian or vertebrate test samples may generally, be obtained from, e.g., bovine, goat, sheep, equine, rabbit, guinea pig, murine, human, feline, monkey, dog, porcine or chicken specimens or subjects.

Test samples can include body fluids, such as urine, whole blood, serum, plasma, semen, cerebrospinal fluid, pus, amniotic fluid, sweat, tears, or semisolid or fluid discharge, e.g., sputum, saliva, lung aspirate, gastrointestinal contents, vaginal or urethral discharge, stool or solid tissue samples, such as a biopsy or chorionic villi specimens. Test samples also include samples collected with swabs from the skin, genitalia, gums or throat. In addition, a representative test sample also includes hair.

In a specific embodiment, a sample of human origin is assayed. In yet another specific embodiment, a serum, plasma, whole blood, sputum, cerebral spinal fluid, amniotic fluid, urine, gastrointestinal contents, hair, saliva, sweat, gum scrapings or tissue from biopsies may be assayed.

In another specific embodiment, an environmental or agricultural sample is assayed. Exemplary environmental or agricultural samples include soil, water, food, dairy, egg and meat samples.

F. Kits

The reagents described above can be packaged in a kit format, preferably with an instruction for using the reagents. The components of the kit can be packaged together in a common container or separate containers, typically including written instructions for performing selected specific embodiments of the methods disclosed herein.

G. Methods of Forming an Aggregate

The present disclosure also provides a method of forming an aggregate with an immuno-aggregator suitable for assaying an analyte, which method comprises: a) providing a plurality of protein components, wherein said protein components do not specifically bind to an analyte to be analyzed; and b) aggregating, without chemical crosslinking or heat treatment, said protein components with an immuno-aggregator that specifically binds to said protein components under suitable conditions to form an aggregate of a defined size suitable for decreasing or eliminating interference in an assay that might otherwise result in a false positive or false negative result.

Any suitable immuno-aggregators, including the immuno-aggregators described in the above Section B.4., can be used in the present method. For example, the immuno-aggregator can be a polyclonal antibody, a monoclonal antibody, or a fragment thereof.

The present methods can be used to generate an aggregate with any suitable size or a mixture of different sizes. For example, the present methods can be used to generate an aggregate having a molecular weight ranging from about 320,000 Dalton to about 100 million Dalton. The present methods also can be used to generate an aggregate with any suitable number of molecules of the protein components per molecule of immuno aggregator. For example, the present methods can be used to generate an aggregate comprising about 10 molecules of the protein components per molecule of immuno aggregator.

The defined size of the aggregate can be achieved via any suitable ways. In one example, the defined size of the aggregate is achieved by controlling the relative quantities of the immuno-aggregator and the protein components, e.g., at a molar ratio of immuno-aggregator to protein component of about 1:1 to about 1:100. In another example, the defined size of the aggregate is achieved by controlling the time and/or the temperature and/or the degree of mixing during the aggregating reaction. For example, the immuno-aggregator and the protein components can be mixed for 5 minutes at 37° C. with a paddle stirrer turning at about 200 rpm and the reaction is halted by gel filtration. In still another example, the defined size of the aggregate is achieved by selecting the formed aggregate with a desired size. The defined size of the aggregate can be selected by any suitable methods such as chromatography, ultrafiltration, flow cytometry, ion exchange or dialysis. Exemplary chromatography methods include molecular weight sieve gel filtration, ion exchange chromatography and hydrophobic interaction chromatography.

The present methods can further comprise removing non-aggregated immuno-aggregator from the formed aggregate. For example, if the protein components are non-specific mouse IgG and the immuno-aggregator is a goat or human anti-mouse-IgG antibody, after the aggregating reaction, the non-aggregated goat or human anti-mouse-IgG immuno aggregator antibody can be removed by interacting them with non-aggregated mouse IgG. The non-aggregated mouse IgG can be contained in a size exclusion chromatography column and the aggregating reaction mixture can pass through the column to remove residue non-aggregated goat or human anti-mouse-IgG antibody. Alternatively, the non-aggregated mouse IgG can be bound to a suitable solid phase, e.g., beads such as sepharose beads. The aggregating reaction mixture can be contacted with a fluid containing the beads to allow binding of the non-aggregated goat or human anti-mouse-IgG immuno aggregator antibody to the beads. The beads can then be separated from the fluid phase by any suitable methods, e.g., centrifugation.

The aggregate formed according to the present methods is also contemplated.

Other features and advantages of the invention will be apparent from the following detailed description, and from the claims.

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

### EXAMPLES

### Example 1

Preparation of Goat Anti-Mouse IgG Antibody as an Immuno Aggregator

Normal mouse IgG was affinity chromatographically purified from normal mouse serum. Goats were immunized with normal mouse IgG with state of art immunization procedures (Stevenson, “Immunisation with antigen coupled to an immunosorbent,” Nature, 247(441):477-8 (1974); Antibody, in The Immunoassay Handbook. Edited by David Wild, 1994, page 50 -54; and Ironside, “Production of anti-human-globulin in goats,” Immunology, 15(4):503-7 (1968)). The immunized goats were bled to derive the goat anti-mouse IgG antiserum. Normal mouse IgG was conjugated via cyanogen bromide coupling to Sepharose 4B agarose gel and packed to a chromatographic column. The goat anti-mouse IgG antiserum was passed through the above column and the goat anti-mouse IgG antibody was affinity purified and concentrated to a final IgG concentration of 10 mg/mL.

### Example 2

Aggregation Between Mouse IgG (Protein Component) and Goat Anti-Mouse IgG Antibody Immuno Aggregator

Three hundred milligrams of normal mouse IgG in a concentration of 10 mg/mL were mixed with 100 mg of goat anti-mouse IgG antibody in a concentration of 10 mg/mL. A light gray color was developed during or after the mixing of these two types of immunoglobulins from two different species. The mixture was incubated at room temperature for 24 hours. The mixture was then filtered through a 0.2-micrometer filter and stored at 2 -8° C.

### Example 3

Immunoassay Procedure

Three capture antibodies of a murine monoclonal anti-human HCG antibody, a goat polyclonal anti-human HCG antibody and a rabbit polyclonal anti-human HCG antibody were mixed and coated onto the surface of each well of a microtiter plate. A murine monoclonal anti-human IGF-I antibody was conjugated with HRP. A volume of 0.2 mL of a human clinical sample was incubated with the three capture antibodies in the well of the microtiter plate at room temperature for 1 hour. The wells were carefully washed and 0.2 mL of HRP conjugated murine monoclonal anti-human IGF-I antibody was added to the wells and incubated at room temperature for 1 hour. The wells were washed and substrate (TMB) for coloring reaction was added. The capture antibodies are specifically against human HCG and the HRP conjugated tracer antibody is specifically against human IGF-I. Therefore, neither human HCG nor IGF-I will specifically bridge both capture and tracer antibodies to generate a colored signal. However, the presence of heterophilic antibody in a clinical sample can form a bridge between the capture and the tracer antibodies resulting in a false positive reaction.

### Example 4

Suppression of Heterophilic Antibody Using the Immunoaggregated Normal Mouse IgG

An amount of heterophilic antibody positive human serum sample was mixed with a volume of 0.01M PBS buffer not containing any mouse IgG and measured using the above immunoassay, which generated a colored signal read at OD450 (>1.5). The same amount of heterophilic antibody positive human serum sample was mixed with the same volume (as 0.01M PBS buffer) of non aggregated normal mouse IgG (10 mg/mL) and measured using the above immunoassay, which generated a colored signal read at OD450 (>1.0). The same amount of the heterophilic antibody positive human serum sample was mixed with the same volume (as 0.01M PBS buffer) of the immuno aggregated normal mouse IgG (10 mg/mL) prepared in Example 2 and measured using the above immunoassay, which generated a colored signal read at OD450 (<0.1). This indicated that the immunoaggregated normal mouse IgG can block the heterophilic antibody activity with very high potency and at an efficiency that is greater when compared to non aggregated normal mouse IgG.

Numerous modifications may be made to the foregoing systems without departing from the basic teachings thereof. Although the present invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. All publications or patent documents cited in this specification are incorporated herein by reference as if each such publication or document was specifically and individually indicated to be incorporated herein by reference.

Citation of the above publications or documents is not intended as an admission that any of the foregoing is pertinent prior art, nor does it constitute any admission as to the contents or date of these publications or documents.

What is claimed is:

1. A reagent for analyzing an analyte, which reagent comprises:
a) an immunoreactant that specifically binds to an analyte in a sample; and
b) an aggregate which suppresses a false positive or a false negative signal caused by an interferent if present in said sample, said aggregate comprising a plurality of protein components, wherein said protein components do not specifically bind to said analyte and said protein components are aggregated together by an immuno-aggregator that specifically binds to said protein components, and wherein said aggregate is formed without chemical crosslinking or heat treatment.

2. The reagent of claim 1, wherein the immunoreactant, the protein component or the immuno-aggregator is a polyclonal antibody, a monoclonal antibody, or a fragment thereof.

3. The reagent of claim 2, wherein the antibody fragment is a Fab or F(ab′)₂ fragment.

4. The reagent of claim 2, wherein the polyclonal antibody or monoclonal antibody is selected from the group consisting of IgA, IgD, IgE, IgG and IgM.

5. The reagent of claim 4, wherein the polyclonal antibody or monoclonal antibody is IgG.

6. The reagent of claim 5, wherein the IgG is selected from the group consisting of IgG₁, IgG₂, IgG₃, and IgG₄.

7. The reagent of claim 1, wherein the plurality of protein components comprise the same species or types of immunoglobulins or different species or types of immunoglobulins.

8. The reagent of claim 7, wherein the different immunoglobulins belong to different types or subtypes of immunoglobulins or immunoglobulins from different species.

9. The reagent of claim 7, wherein the different immunoglobulins are derived from different vertebrate species.

10. The reagent of claim 1, wherein the plurality of protein components comprise IgGs.

11. The reagent of claim 1, wherein the plurality of protein components comprise IgG and Fab or F(ab′)₂ fragment.

12. The reagent of claim 1, wherein the immuno-aggregator comprises IgG, Fab or F(ab′)₂ fragment.

13. The reagent of claim 1, wherein the immunoreactant, the protein component or the immuno-aggregator is derived from a mammal or a vertebrate.

14. The reagent of claim 13, wherein the mammal or a vertebrate is selected from the group consisting of bovine, goat, sheep, equine, rabbit, guinea pig, murine, human, feline, porcine, monkey, dog and chicken.

15. The reagent of claim 1, wherein the analyte and the immunoreactant are derived from the same or different species.

16. The reagent of claim 1, wherein the immunoreactant and the immuno-aggregator are derived from the same or different species.

17. The reagent of claim 1, wherein the analyte is derived from a human sample, the protein components are murine proteins and the immuno-aggregator is a goat anti-murine protein antibody.

18. The reagent of claim 1, wherein the aggregate has a concentration ranging from about 0.1 μg/ml to about 5,000 μg/ml.

19. The reagent of claim 1, wherein the aggregate has a molecular weight of at least about 320,000 daltons.

20. The reagent of claim 1, wherein the aggregate has a molecular weight ranging from about 320,000 Dalton to about 100 million Dalton.

21. The reagent of claim 1, wherein the immunoaggregate further comprises a water soluble macromolecule.

22. The reagent of claim 21, wherein the water soluble macromolecule is selected from the group consisting of a water soluble protein, a water soluble polysaccharide and a water soluble polymer.

23. The reagent of claim 1, wherein the immunoreactant carries a label.

24. The reagent of claim 1, wherein the immunoreactant is attached to a surface suitable for conducting an immunoassay.

25. The reagent of claim 1, which comprises two different immunoreactants that can sandwich an analyte.

26. The reagent of claim 25, wherein both of the two different immunoreactants are intact IgGs.

27. The reagent of claim 26, wherein the aggregate comprises intact IgGs.

28. The reagent of claim 25, wherein one of the two different immunoreactants is an intact IgG and the other immunoreactant is a Fab or F(ab′)₂ fragment.

29. The reagent of claim 28, wherein the aggregate comprises intact IgGs and Fab or F(ab′)₂ fragments.

30. The reagent of claim 25, wherein the two different immunoreactants are murine anti-human analyte antibodies or fragments thereof, the protein components in the aggregate are non-specific murine antibodies or fragments thereof, and the immuno-aggregator in the aggregate is a goat anti-murine antibody or fragments thereof.

31. The reagent of claim 1, which comprises an antigen and an immunoreactant that can sandwich an antibody analyte.

32. The reagent of claim 1, wherein the molar ratio between the protein components in the aggregate and the immuno-aggregator in the aggregate is more than 1.

33. The reagent of claim 1, wherein the molar ratio between the protein components in the aggregate and the immuno-aggregator in the aggregate is less than 1.

34. The reagent of claim 1, which further comprises a reaction accelerator, a detergent, or a stabilizer.

35. The reagent of claim 1, which further comprises an analyte bound to the immunoreactant in the reagent.

36. A kit for analyzing an analyte, which kit comprises a reagent of claim 1 in a container.

37. The kit of claim 36, wherein the immunoreactant and the aggregate are comprised in the same or different containers.

38. The kit of claim 36, which further comprises a buffer or an instruction for using the reagent to analyze an analyte.

39. A method for analyzing an analyte, which method comprises:
a) contacting an analyte in a sample with a reagent of claim 1 under suitable conditions to allow binding between said analyte, if present in said sample, and said immunoreactant in said reagent, while suppressing a false positive or a false negative signal caused by an interferent, if present in said sample, via an interaction between said interferent and said aggregate in said reagent, or via an interaction between said aggregate and a non-specific binding site for said interferent; and
b) assessing binding between said analyte and said immunoreactant to analyze the presence or amount of said analyte in said sample.

40. The method of claim 39, wherein the analyte is selected from the group consisting of a cell, a cellular organelle, a virus, a molecule and an aggregate or complex thereof.

41. The method of claim 40, wherein the cell is selected from the group consisting of an animal cell, a plant cell, a fungus cell, a bacterium cell, a recombinant cell and a cultured cell.

42. The method of claim 40, wherein the cellular organelle is selected from the group consisting of a nuclei, a mitochondrion, a chloroplast, a ribosome, an ER, a Golgi apparatus, a lysosome, a proteasome, a secretory vesicle, a vacuole and a microsome.

43. The method of claim 40, wherein the molecule is selected from the group consisting of an inorganic molecule, an organic molecule and a complex thereof.

44. The method of claim 43, wherein the organic molecule is selected from the group consisting of an amino acid, a peptide, a protein, a nucleoside, a nucleotide, an oligonucleotide, a nucleic acid, a vitamin, a monosaccharide, an oligosaccharide, a carbohydrate, a lipid and a complex thereof.

45. The method of claim 39, wherein the analyte is selected from the group consisting of a hormone, a cancer marker, a steroid, a sterol, a pharmaceutical compound, a metabolite of a pharmaceutical compound and a complex thereof.

46. The method of claim 39, wherein the sample is mammalian or vertebrate sample.

47. The method of claim 46, wherein the mammal or vertebrate is selected from the group consisting of bovine, goat, sheep, equine, rabbit, guinea pig, murine, human, feline, monkey, dog, porcine, dog and chicken.

48. The method of claim 39, wherein the sample is a clinical sample.

49. The method of claim 48, wherein the clinical sample is selected from the group consisting of serum, plasma, whole blood, sputum, cerebral spinal fluid, amniotic fluid, urine, gastrointestinal contents, hair, saliva, sweat, gum scrapings and tissue from biopsies.

50. The method of claim 48, wherein the clinical sample is a human clinical sample.

51. The method of claim 39, wherein the sample is a body fluid sample.

52. The method of claim 39, wherein the interferent is selected from the group consisting of a heterophilic antibody, a rheumatoid factor, a lipoprotein, a fibrin, a clotting factor, an IgE, a human antibody to allergens, a human anti-mouse immunoglobulin, a human anti-goat immunoglobulin, a human anti-bovine immunoglobulin, a human anti-dog immunoglobulin and a human anti-rabbit immunoglobulin.

53. The method of claim 39, wherein the aggregate in the reagent substantially suppresses a false positive or a false negative signal caused by an interferent, if present in said sample.

54. The method of claim 39, wherein the binding between the analyte and the immunoreactant is assessed by a sandwich or competitive assay format.

55. The method of claim 39, wherein the binding between the analyte and the immunoreactant is assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, chemiluminescence assay, lateral flow immunoassay, μ-capture assay, inhibition assay, energy transfer assay, avidity assay, turbidometric immunoassay and time resolved amplified cryptate emission (TRACE) assay.

56. The method of claim 39, wherein the interference to be suppressed is a false-positive result.

57. The method of claim 39, wherein the interference to be suppressed is a false-negative result.

58. The method of claim 39, wherein the molar ratio between the protein components in the aggregate and the immuno-aggregator in the aggregate is more than 1.

59. The method of claim 39, wherein the molar ratio between the protein components in the aggregate and the immuno-aggregator in the aggregate is less than 1.

60. The method of claim 39, wherein the immunoreactant and the protein components in the aggregate are derived from the same species.

61. The method of claim 39, wherein the immunoreactant is an antibody, the interferent, if present, would bind with said immunoreactant to generate a false negative result, and the aggregate interacts with said interferent to suppress said false negative result.

62. The method of claim 61, wherein the immunoreactant is a non-human antibody, and the interferent is human heterophilic antibody.

63. The method of claim 62, wherein the analyte to be analyzed is a human antibody.

64. The method of claim 39, wherein the analyte to be analyzed is an antibody, the interferent, if present, would bind with at least two assay antibodies to generate a false positive result, and the aggregate interacts with said interferent to suppress said false positive result.

65. The method of claim 64, wherein the analyte to be analyzed is a human antibody, and the interferent is human heterophilic antibody.

66. The method of claim 39, wherein the analyte to be analyzed is an antibody, the interferent, if present, would bind with at least an assay antibody and at least an assay antigen to generate a false positive result, and the aggregate interacts with said interferent to suppress said false positive result.

67. The method of claim 66, wherein the analyte to be analyzed is a human antibody, and the interferent is human heterophilic antibody.

68. A method of forming an aggregate suitable for assaying an analyte, which method comprises:
a) providing a plurality of protein components, wherein said protein components do not specifically bind to an analyte to be analyzed; and
b) aggregating, without chemical crosslinking or heat treatment, said protein components with an immuno-aggregator that specifically binds to said protein components under suitable conditions into an aggregate of a defined size suitable for assaying said analyte.

69. The method of claim 68, wherein the immuno-aggregator is a polyclonal antibody, a monoclonal antibody, or a fragment thereof.

70. The method of claim 68, wherein the aggregate has a molecular weight ranging from about 320,000 Dalton to about 100 million Dalton.

71. The method of claim 68, wherein the aggregate comprises about 10 molecules of the protein components per molecule of the immuno-aggregator.

72. The method of claim 68, wherein the defined size of the aggregate is achieved by controlling the relative quantities of the immuno-aggregator and the protein components.

73. The method of claim 68, wherein the defined size of the aggregate is achieved by controlling the time and/or the temperature of the aggregating reaction.

74. The method of claim 68, wherein the defined size of the aggregate is achieved by selecting the formed aggregate with a desired size.

75. The method of claim 74, wherein the formed aggregate with a desired size is selected by chromatography, ultrafiltration or dialysis.

76. The method of claim 75, wherein the chromatography is molecular weight sieve gel filtration, ion exchange chromatography or hydrophobic interaction chromatography.

77. The method of claim 68, further comprising removing non-aggregated immuno-aggregator from the formed aggregate.

78. The aggregate formed according to the method of claim 68.
